SEQUENCE LISTING (17 sequences)

SEQ ID NO: 1 - DNA, 1206 bp, Solanum tuberosum
atggccttttctgctggaaaatcttatgcattttcaacaaattttgatcccttgcaacaaaatttatggggttctaagcctcctatttcatcattatccatcaaagatatcgacttcaaacaatgtgataagcataacatcttgtctaaaaaacctctatacatttcagcagtactaagtggatttggacatgctgatgaatcaaaagagttcaaatctagggaccctttagtccaatgcaatgcatatgaagctagtcaaccacaatcgataccaatcgacattgagtttggccaagaagctcaggctgctgctactcagaagctcaagattggactctattttgcaacatggtgggctttgaatgttgttttcaatatttacaacaagaaggttttgaatgcatttccatttccatggcttacttctactctttctctggctgctggctctcttatgatgttggtttcttgggctactaagattgctgaaactccaaagactgactttgatttttggaaagccttgtttcctgttgctgtggctcacacaattggtcatgtggctgcaacagtgagcatgtcaaaagttgctgtttcattcactcatattattaagagtggagagcctgcttttagtgttttggtttcaagattgttaggtgaaactttcccattgccagtgtacctttcacttttgccaattattggtggttgtggacttgctgctattactgaactcaacttcaatctaataggttttatgggggcaatgatatcaaacttggcatttgtattcagaaacatattttcaaagaaaggcatgaaggggaaatcagttggagggatgaattactatgcttgtttatcaatgatgtctctattgattctgattccctttgctattgctgtggagggtccacaagtatgggcacttggatggcagaatgcagtctcccaaattggcccaaattttatatggtgggtggtggctcaaagtgtgttctatcacttgtacaatcaagtctcctacatgtccctaaatgagatctccccattgacatttagtattggtaacactatgaagagaatttctgtcatagtctcatccatcatcatctttcaaattccaattcaaccaatcaatgcccttggtgctgctattgcaatcttgggaactttcctctactctcaggctaaacagtaa

SEQ ID NO: 2 - protein, 401 aa, Solanum tuberosum
MAFSAGKSYAFSTNFDPLQQNLWGSKPPISSLSIKDIDFKQCDKHNILSKKPLYISAVLSGFGHADESKEFKSRDPLVQCNAYEASQPQSIPIDIEFGQEAQAAATQKLKIGLYFATWWALNVVFNIYNKKVLNAFPFPWLTSTLSLAAGSLMMLVSWATKIAETPKTDFDFWKALFPVAVAHTIGHVAATVSMSKVAVSFTHIIKSGEPAFSVLVSRLLGETFPLPVYLSLLPIIGGCGLAAITELNFNLIGFMGAMISNLAFVFRNIFSKKGMKGKSVGGMNYYACLSMMSLLILIPFAIAVEGPQVWALGWQNAVSQIGPNFIWWVVAQSVFYHLYNQVSYMSLNEISPLTFSIGNTMKRISVIVSSIIIFQIPIQPINALGAAIAILGTFLYSQAKQ

SEQ ID NO: 3 - DNA, 1899 bp, Solanum tuberosum
atggctatggagagtgcattgacatccacacgagtttcaattccatcgttgtgttctgggatcagttcatctcatcatcaccatagatccttatcatttctcaatttccccaaattgtcttcattcaaatactcttttcgaacaatttcacctgttcctttcgttgtcagtgcatcctctgtttctccatcatctccttctacatctgttgcccaatctcaggatctcaagattaaatcagttcctaccaagcctattgaaggtcaaaagaccggtaccagtggtctacgcaaaaaggttaaagtgtttatgcaggacaattaccttgccaattggatacaggcattgtttaattctttgccgttagaggattataagaatggactcttggtcttaggaggtgatggtcgatatttcaatcgagaagctgcacagataatcattaaaattgctgcgggtaacggtgttggtaaaatattggttggcaaggacggtatactgtctactcaagctgtgtctgctgtgatacggaaaagagaggcaaatggtggctttataatgagtgcaagccataaccctggtggtcccgagtatgattggggtatcaagttcaattatagcagcggccaaccagctcccgagtctatcacggacaagatctatggaaataccctttctatttctgaaataaaaattgctgacattcctgatgttgatctttctcaacttggagttacaaaatatggaaattttagtgtggaggtggttgaccctgtggctgattatttggaactaatggaaaatgtatttgatttttcactcattagaagtcttgtttcacgaccagatttcaggtttgtatttgatgccatgcatgcagttacaggggcttatgcaaagcccatttttgttgacaaactaggagctagcctggagtctattgcaaatggagtgccactggaagattttggacatggtcatccagaccctaaccttacgtatgctgaggatttggtgaatatcctgtatggagagaatggacctgactttggtgctgcaagcgatggagatggtgatagaaacatgattcttggaagaagcttttttgttactccatctgattctgttgcaattattgctgcccaatgccaatatgctatccattatttccagtccgggcctaagggtttggctcgatcaatgcccactagcggttctctagatcgcgttgcccaaaagctaaacctccccttttttgaggtgccaactggttggaaattctttggtaatctaatggatgctggaaaattgtcaatctgtggcgaagaaagttttgggactggttctgaccacatccgtgaaaaagatggtatatgggctgttttagcttggctttcaatacttgcatatcggaacaaggacaaaaaatcaggggagaaattggtttctgttgctgatgttgtgaaggatcattgggccacttatggaaggaacttcttttcgagatatgactatgaggagtgtgaatcagaaggagcaaacaacatgattgagtatcttcgggatttgatttctaaaagcaaggctggtgataagtatggaagttatagcctagattttgctgatgacttcgcctatactgatcctgtcgatggaagtgttgcatccaaacagggggttcgctttgtttttagtgatgggtcacggatcatctttcgactatcgggtactggttctgctggtgcaacagtcagaatatatatcgaacagtttgagccggatgtttccaaacacgacatggatgctcaaattgcattgaaaccattgatagatctcgctctttctgtatcaaagctaaaggacttcaccggaagagaaaagcctactgtcatcacataa

SEQ ID NO: 4 - protein, 632 aa, Solanum tuberosum
MAMESALTSTRVSIPSLCSGISSSHHHHRSLSFLNFPKLSSFKYSFRTISPVPFVVSASSVSPSSPSTSVAQSQDLKIKSVPTKPIEGQKTGTSGLRKKVKVFMQDNYLANWIQALFNSLPLEDYKNGLLVLGGDGRYFNREAAQIIIKIAAGNGVGKILVGKDGILSTQAVSAVIRKREANGGFIMSASHNPGGPEYDWGIKFNYSSGQPAPESITDKIYGNTLSISEIKIADIPDVDLSQLGVTKYGNFSVEVVDPVADYLELMENVFDFSLIRSLVSRPDFRFVFDAMHAVTGAYAKPIFVDKLGASLESIANGVPLEDFGHGHPDPNLTYAEDLVNILYGENGPDFGAASDGDGDRNMILGRSFFVTPSDSVAIIAAQCQYAIHYFQSGPKGLARSMPTSGSLDRVAQKLNLPFFEVPTGWKFFGNLMDAGKLSICGEESFGTGSDHIREKDGIWAVLAWLSILAYRNKDKKSGEKLVSVADVVKDHWATYGRNFFSRYDYEECESEGANNMIEYLRDLISKSKAGDKYGSYSLDFADDFAYTDPVDGSVASKQGVRFVFSDGSRIIFRLSGTGSAGATVRIYIEQFEPDVSKHDMDAQIALKPLIDLALSVSKLKDFTGREKPTVIT

SEQ ID NO: 5 - DNA, 1563 bp, Solanum tuberosum
atgaaatcgacggtccatttggggagagtgagcactggtggctttaacaatggagagaaggagtttttcggggagaagatcagagggagtttgaacagcaatctcaggattaatcagttgtcgaaaagtttgaaacttgagaagaaggagaacaagatcaaacctggggttgcttactctgtgatcactactgaaaatgacacacagactgtgttcgtagatatgccacgtcttgagagacgccgggcaaatccaaaggatgtggctgcagtcatactaggaggaggagaagggaccaagttattcccacttacaagtagaactgcaacccctgctgttccggttggaggatgctacaggctaatagacatcccaatgagcaactgtatcaacagtgctattaacaagatttttgtgctgacacagtacaattctgctcccctgaatcgtcacattgctcgaacatattttggcaatggtgtgagctttggagatggatttgtcgaggtactagctgcaactcagacacctggggaagcaggaaaaaaatggtttcaaggaacagcagatgctgttagaaaatttatatgggtttttgaggacgctaagaacaagaatattgaaaatatccttgtattatctggggatcatctttataggatggattatatggagttggtgcagaaccatattgacaggaatgctgatattactctttcatgtgcaccagctgaggacagccgagcatcagattttgggctggtcaagattgacagcagaggcagagttgtccagtttgctgaaaaaccaaaaggttttgatcttaaagcaatgcaagtagatactactcttgttggattatctccacaagatgcgaagaaatccccctatattgcttcaatgggagtttatgtattcaagacagatgtattgttgaagctcttgaaatggagctatcccacttctaatgattttggctctgaaattataccagcagctattgacgattacaatgtccaagcatacattttcaaagactattgggaggacattggaacaattaaatctttttataatgctagcttggcactcacacaagagtttccagagttccaattttacgatccaaaaacacctttttacacatctcctaggttccttccaccaaccaagatagacaattgcaagattaaggatgccataatctctcatggatgtttcttgcgagattgttctgtggaacactccatagtgggtgaaagatcgcgcttagattgtggtgttgaactgaaggatactttcatgatgggagcagactactaccaaacagaatctgagattgcctccctgttagcagaggggaaagtaccgattggaattggggaaaatacaaaaataaggaaatgtatcattgacaagaacgcaaagataggaaagaatgtttcaatcataaataaagacggtgttcaagaggcagaccgaccagaggaaggattctacatacgatcaggcataatcattatattagagaaagccacaattagagatggaacagtcatatga

SEQ ID NO: 6 - protein, 520 aa, Solanum tuberosum
MKSTVHLGRVSTGGFNNGEKEFFGEKIRGSLNSNLRINQLSKSLKLEKKENKIKPGVAYSVITTENDTQTVFVDMPRLERRRANPKDVAAVILGGGEGTKLFPLTSRTATPAVPVGGCYRLIDIPMSNCINSAINKIFVLTQYNSAPLNRHIARTYFGNGVSFGDGFVEVLAATQTPGEAGKKWFQGTADAVRKFIWVFEDAKNKNIENILVLSGDHLYRMDYMELVQNHIDRNADITLSCAPAEDSRASDFGLVKIDSRGRVVQFAEKPKGFDLKAMQVDTTLVGLSPQDAKKSPYIASMGVYVFKTDVLLKLLKWSYPTSNDFGSEIIPAAIDDYNVQAYIFKDYWEDIGTIKSFYNASLALTQEFPEFQFYDPKTPFYTSPRFLPPTKIDNCKIKDAIISHGCFLRDCSVEHSIVGERSRLDCGVELKDTFMMGADYYQTESEIASLLAEGKVPIGIGENTKIRKCIIDKNAKIGKNVSIINKDGVQEADRPEEGFYIRSGIIIILEKATIRDGTVI

SEQ ID NO: 7 - DNA, 1896 bp, Solanum tuberosum
atggaaggtgttttacaaacaagagggcttctttctttgccttctaaacccaaaatcaaggctttttacccattgcctcaagggggtctaaggaacagattcaattctttaagtagtttaaagcctaatcctcttaatggggtttctttatcttcaaatgggtttcaaaaagttcaaggctttgacacaaagcctcagttgtttggccaaaagaagaggtgttttccaatatgcaaagctgaggctgctgctgctgctggtgcagctgatggacagccactttttgttgaaaaggagcaacctaagtttatggggattgaacttgtgacccttaagaaaattataccacttggggcgatgttcttttgtattctgtttaattatacaatccttagggatactaaggatgtgttggttgtaacagctaaagggtccagtgctgagattatccctttcttgaaaacttgggtgaatttgcctatggctattggattcatgcttttgtacacaaagttggctaatgtgttgtcaaaggaggctcttttttatactgttatacttccttttattgcattctttggggcgtttggttttgttttgtatcctcttagcaattactttcaccctacagcttttgctgataagcttctcaatacccttggtccaagatttcttggaccaattgctattctgaggatctggagtttctgcttgttctatgtcatggctgagctttggggaagtgtggtggtttcagtactcttttggggatttgctaatcagattacaactatcgatgaggctaagagattctatcctttgtttggacttggagcgaatgttgctcttattttctctggtcgcacagtgaagtacttttctagcttgagaagctctttaggtcctggagttgatggttgggctatctccctgaaaggaatgatgagcattgttgtgatgatgggtggggcaatctgtttcttttactggtgggtgaatagaaatgttgctctcccaactcgtagcaagaagaagaaggtaaaacctaacatgaccacaatggagagcttgaagttcttggtctcttcaaaatatatcagggatcttgccacattggttgtagcatatggcattagtatcaaccttgttgaagttacatggaagtcaaagctcaaagctcagttcccaagccccaatgaatactcctcattcatgggtgacttctcaactgctactggaatagcaactttcacaatgatgttgttaagtcaatggattttcgacaagtatgggtggggagcagcagccaagataacacctacagtcttgctcctaaccggagttggtttcttctccctgcttttgtttggtgcccctctagcacctactcttgcgaagtttggaatgactcctcttctagcagctgtctatgtgggtgcaatgcagaacattttcagtaagagtgcaaagtatagtttgtttgacccctgcaaagaaatggcctacattcctttggatgaggacaccaaggttaaagggaaggcagcaatcgatgttgtctgcaatccactgggaaagtctggaggagctttgatacaacaattcatgattttgacttttggttcacttgccagctcgacaccctacctcggcggtgtgctcttagtaattgttcttgcatggttgggagcagccaagtctttggatggacagttcactcaattacgccaagaagaagatcttgagaaggaaatggagagagcatcgttgaagatccctgtcgtgtctcaaaatgaaaatggaaatggtcctctctcaagtgggtcatcactaaatcccgctggaggtgactctaccaacgcttcatcagaaccctcctccccaaggagcctgtaa

SEQ ID NO: 8 - protein, 631 aa, Solanum tuberosum
MEGVLQTRGLLSLPSKPKIKAFYPLPQGGLRNRFNSLSSLKPNPLNGVSLSSNGFQKVQGFDTKPQLFGQKKRCFPICKAEAAAAAGAADGQPLFVEKEQPKFMGIELVTLKKIIPLGAMFFCILFNYTILRDTKDVLVVTAKGSSAEIIPFLKTWVNLPMAIGFMLLYTKLANVLSKEALFYTVILPFIAFFGAFGFVLYPLSNYFHPTAFADKLLNTLGPRFLGPIAILRIWSFCLFYVMAELWGSVVVSVLFWGFANQITTIDEAKRFYPLFGLGANVALIFSGRTVKYFSSLRSSLGPGVDGWAISLKGMMSIVVMMGGAICFFYWWVNRNVALPTRSKKKKVKPNMTTMESLKFLVSSKYIRDLATLVVAYGISINLVEVTWKSKLKAQFPSPNEYSSFMGDFSTATGIATFTMMLLSQWIFDKYGWGAAAKITPTVLLLTGVGFFSLLLFGAPLAPTLAKFGMTPLLAAVYVGAMQNIFSKSAKYSLFDPCKEMAYIPLDEDTKVKGKAAIDVVCNPLGKSGGALIQQFMILTFGSLASSTPYLGGVLLVIVLAWLGAAKSLDGQFTQLRQEEDLEKEMERASLKIPVVSQNENGNGPLSSGSSLNPAGGDSTNASSEPSSPRSL

SEQ ID NO: 9 - DNA, 873 bp, Solanum tuberosum
atggcggcggcaagagtcatggtatcagccagcaacactttaacggcttcgctcctatcgaaaggtccattacgaaagcccaacaatttcagcctttgcttcagaaacggaccggtgcaaaagaggcgtcttttcacgtgcagtgcaatctacaatccacagattcagactatagaagaaggacagcccgaaaccctagattaccgtgtgttttttgcagacaattccggcaaaaagatatccccttggcatgacataccactacatttgggtgatggtgttttcaattttgttgttgagatccccaaagaatcaagtgcaaagatggaagttgctacagatgagcaacacactccaattaaacaagacacgaagaaggggaaacttcgatactatccatataatattcactggaactatggattgcttcctcaaacatgggaagatcctacttttgcgaacaccgaagttgagggggcatttggtgataatgaccctgttgatgttgttgagattggggaaagccgaggtaaaattggccaggttctgaaggtcaaacctttagctgctctggcaatgattgatgaaggagaacttgactggaaaatagttgctatttcactagatgatccaagagcttcacttgttaatgatgttgatgatgtggaaaaacattttccgggcactctcacagcaatcagggattggtttagagactataagatacctgatggaaaacctgccaataggtttgctcttggcaacaagccagcaaacaaggattacgctcttaagattattacggaaaccaatgaatcttgggcaaagctggtcaagagatcaatcgctgctggtgagctttcacttgtataa

SEQ ID NO: 10 - protein, 290 aa, Solanum tuberosum
MAAARVMVSASNTLTASLLSKGPLRKPNNFSLCFRNGPVQKRRLFTCSAIYNPQIQTIEEGQPETLDYRVFFADNSGKKISPWHDIPLHLGDGVFNFVVEIPKESSAKMEVATDEQHTPIKQDTKKGKLRYYPYNIHWNYGLLPQTWEDPTFANTEVEGAFGDNDPVDVVEIGESRGKIGQVLKVKPLAALAMIDEGELDWKIVAISLDDPRASLVNDVDDVEKHFPGTLTAIRDWFRDYKIPDGKPANRFALGNKPANKDYALKIITETNESWAKLVKRSIAAGELSLV

SEQ ID NO: 11 - protein, 575 aa, Solanum tuberosum
ASSVSPSSPSTSVAQSQDLKIKSVPTKPIEGQKTGTSGLRKKVKVFMQDNYLANWIQALFNSLPLEDYKNGLLVLGGDGRYFNREAAQIIIKIAAGNGVGKILVGKDGILSTQAVSAVIRKREANGGFIMSASHNPGGPEYDWGIKFNYSSGQPAPESITDKIYGNTLSISEIKIADIPDVDLSQLGVTKYGNFSVEVVDPVADYLELMENVFDFSLIRSLVSRPDFRFVFDAMHAVTGAYAKPIFVDKLGASLESIANGVPLEDFGHGHPDPNLTYAEDLVNILYGENGPDFGAASDGDGDRNMILGRSFFVTPSDSVAIIAAQCQYAIHYFQSGPKGLARSMPTSGSLDRVAQKLNLPFFEVPTGWKFFGNLMDAGKLSICGEESFGTGSDHIREKDGIWAVLAWLSILAYRNKDKKSGEKLVSVADVVKDHWATYGRNFFSRYDYEECESEGANNMIEYLRDLISKSKAGDKYGSYSLDFADDFAYTDPVDGSVASKQGVRFVFSDGSRIIFRLSGTGSAGATVRIYIEQFEPDVSKHDMDAQIALKPLIDLALSVSKLKDFTGREKPTVIT

SEQ ID NO: 12 - DNA, 1659 bp, Solanum tuberosum
gctgaggctgctgctgctgctggtgcagctgatggacagccactttttgttgaaaaggagcaacctaagtttatggggattgaacttgtgacccttaagaaaattataccacttggggcgatgttcttttgtattctgtttaattatacaatccttagggatactaaggatgtgttggttgtaacagctaaagggtccagtgctgagattatccctttcttgaaaacttgggtgaatttgcctatggctattggattcatgcttttgtacacaaagttggctaatgtgttgtcaaaggaggctcttttttatactgttatacttccttttattgcattctttggggcgtttggttttgttttgtatcctcttagcaattactttcaccctacagcttttgctgataagcttctcaatacccttggtccaagatttcttggaccaattgctattctgaggatctggagtttctgcttgttctatgtcatggctgagctttggggaagtgtggtggtttcagtactcttttggggatttgctaatcagattacaactatcgatgaggctaagagattctatcctttgtttggacttggagcgaatgttgctcttattttctctggtcgcacagtgaagtacttttctagcttgagaagctctttaggtcctggagttgatggttgggctatctccctgaaaggaatgatgagcattgttgtgatgatgggtggggcaatctgtttcttttactggtgggtgaatagaaatgttgctctcccaactcgtagcaagaagaagaaggtaaaacctaacatgaccacaatggagagcttgaagttcttggtctcttcaaaatatatcagggatcttgccacattggttgtagcatatggcattagtatcaaccttgttgaagttacatggaagtcaaagctcaaagctcagttcccaagccccaatgaatactcctcattcatgggtgacttctcaactgctactggaatagcaactttcacaatgatgttgttaagtcaatggattttcgacaagtatgggtggggagcagcagccaagataacacctacagtcttgctcctaaccggagttggtttcttctccctgcttttgtttggtgcccctctagcacctactcttgcgaagtttggaatgactcctcttctagcagctgtctatgtgggtgcaatgcagaacattttcagtaagagtgcaaagtatagtttgtttgacccctgcaaagaaatggcctacattcctttggatgaggacaccaaggttaaagggaaggcagcaatcgatgttgtctgcaatccactgggaaagtctggaggagctttgatacaacaattcatgattttgacttttggttcacttgccagctcgacaccctacctcggcggtgtgctcttagtaattgttcttgcatggttgggagcagccaagtctttggatggacagttcactcaattacgccaagaagaagatcttgagaaggaaatggagagagcatcgttgaagatccctgtcgtgtctcaaaatgaaaatggaaatggtcctctctcaagtgggtcatcactaaatcccgctggaggtgactctaccaacgcttcatcagaaccctcctccccaaggagcctgtaa

SEQ ID NO: 13 - protein, 552 aa, Solanum tuberosum
AEAAAAAGAADGQPLFVEKEQPKFMGIELVTLKKIIPLGAMFFCILFNYTILRDTKDVLVVTAKGSSAEIIPFLKTWVNLPMAIGFMLLYTKLANVLSKEALFYTVILPFIAFFGAFGFVLYPLSNYFHPTAFADKLLNTLGPRFLGPIAILRIWSFCLFYVMAELWGSVVVSVLFWGFANQITTIDEAKRFYPLFGLGANVALIFSGRTVKYFSSLRSSLGPGVDGWAISLKGMMSIVVMMGGAICFFYWWVNRNVALPTRSKKKKVKPNMTTMESLKFLVSSKYIRDLATLVVAYGISINLVEVTWKSKLKAQFPSPNEYSSFMGDFSTATGIATFTMMLLSQWIFDKYGWGAAAKITPTVLLLTGVGFFSLLLFGAPLAPTLAKFGMTPLLAAVYVGAMQNIFSKSAKYSLFDPCKEMAYIPLDEDTKVKGKAAIDVVCNPLGKSGGALIQQFMILTFGSLASSTPYLGGVLLVIVLAWLGAAKSLDGQFTQLRQEEDLEKEMERASLKIPVVSQNENGNGPLSSGSSLNPAGGDSTNASSEPSSPRSL

SEQ ID NO: 14 - DNA, 729 bp, Solanum tuberosum
gcaatctacaatccacagattcagactatagaagaaggacagcccgaaaccctagattaccgtgtgttttttgcagacaattccggcaaaaagatatccccttggcatgacataccactacatttgggtgatggtgttttcaattttgttgttgagatccccaaagaatcaagtgcaaagatggaagttgctacagatgagcaacacactccaattaaacaagacacgaagaaggggaaacttcgatactatccatataatattcactggaactatggattgcttcctcaaacatgggaagatcctacttttgcgaacaccgaagttgagggggcatttggtgataatgaccctgttgatgttgttgagattggggaaagccgaggtaaaattggccaggttctgaaggtcaaacctttagctgctctggcaatgattgatgaaggagaacttgactggaaaatagttgctatttcactagatgatccaagagcttcacttgttaatgatgttgatgatgtggaaaaacattttccgggcactctcacagcaatcagggattggtttagagactataagatacctgatggaaaacctgccaataggtttgctcttggcaacaagccagcaaacaaggattacgctcttaagattattacggaaaccaatgaatcttgggcaaagctggtcaagagatcaatcgctgctggtgagctttcacttgtataa

SEQ ID NO: 15 - protein, 242 aa, Solanum tuberosum
AIYNPQIQTIEEGQPETLDYRVFFADNSGKKISPWHDIPLHLGDGVFNFVVEIPKESSAKMEVATDEQHTPIKQDTKKGKLRYYPYNIHWNYGLLPQTWEDPTFANTEVEGAFGDNDPVDVVEIGESRGKIGQVLKVKPLAALAMIDEGELDWKIVAISLDDPRASLVNDVDDVEKHFPGTLTAIRDWFRDYKIPDGKPANRFALGNKPANKDYALKIITETNESWAKLVKRSIAAGELSLV

SEQ ID NO: 16 - DNA, 20 bp, Artificial Sequence
  primer nptIIf
cctgtcatctcaccttgctc

SEQ ID NO: 17 - DNA, 20 bp, Artificial Sequence
  primer nptIIr
agtcccgctcagaagaactc

(19) United States
(12) Patent Application Publication
(10) Pub. No.: US 2016/0312237 A1
(43) Pub. Date: Oct. 27, 2016

(54) Transgenic Plant

(71) Applicant: Sveriges Stärkelseproducenter, Förening UPA, Kristianstad (SE)

(72) Inventors: Olof WIKSTRÖM, Kristianstad (SE); Åke WIRTÉN, Karlshamn (SE); Per HOFVANDER, Bjärred (SE); Mariette ANDERSSON, Lund (SE); Helle TURESSON, Dösjebro (SE)

(73) Assignee: Sveriges Stärkelseproducenter, Förening UPA, Kristianstad (SE)

(21) Appl. No.: 14/915,146

(22) PCT Filed: Aug. 29, 2014
(86) PCT No.: PCT/SE2014/050997

§ 371 Date: Feb. 26, 2016

(30) Foreign Application Priority Data
Aug. 29, 2013 (SE) 1350996-3

(51) Int. Cl. C12N 15/82; A01H 5/06; C08B 30/04

(52) U.S. Cl. CPC C12N 15/8245; C08B 30/04; A01H 5/06

(57) ABSTRACT

The present invention relates to plant cells and plants, which are genetically modified, whereby the genetic modification leads to an alteration of storage compound deposition in Beta vulgaris tap-root, such as sugar beet tap-root or fodder beet tap-root. By the alteration the tap-root of the plants accumulates starch in comparison with the corresponding wild type plant tap-root. In addition, the present invention concerns means and methods for the manufacture of such plant cells and plants. The present invention also concerns the starches synthesised from the tap-root of these plant, methods for manufacturing these starch. Furthermore, the present invention also relates to nucleic acids, coding the genes involved in the synthesis of starch, vectors, host cells, plant cells, and plants containing such nucleic acid molecules.

### FIELD OF INVENTION

The present invention relates to plant cells and plants, which are genetically modified, whereby the genetic modification leads to an alteration of storage compound deposition in Beta vulgaris tap-root, such as sugar beet tap-root or fodder beet tap-root. By the alteration, the tap-root of the plants accumulates starch in comparison with the corresponding wild type plant tap-root that almost exclusively accumulates sucrose. In addition, the present invention concerns means and methods for the manufacture of such plant cells and plants. The present invention also concerns the starches synthesised in the tap-root of these plant and methods for manufacturing these starches. Furthermore, the present invention also relates to functions and corresponding nucleic acids, coding for genes involved in and facilitating the synthesis of starch, vectors, host cells, plant cells, and plants containing such nucleic acid molecules.

### BACKGROUND OF INVENTION

Starch is the main extracted storage compound from crops harvested in agriculture in the world. The main crops used for starch production are maize, wheat, potato and cassava. Potato and cassava are examples of important tuber or root crops for starch production.

In Europe potato is one of the important starch crops with almost 2 million tons of extracted starch produced each year. Mainly this is a northern European operation with main countries for production being Germany and the Netherlands. Another crop of importance for extraction of materials is sugar beet where the main extracted product is sucrose. Since sucrose essentially is the basic export product from photosynthesis which in potato is converted to starch in the tubers it could also be possible to transfer sugar beet into a starch crop. In comparison to potato, sugar beet has a higher productivity, need a lower input of water and chemicals, are handled as a regular seed crop in comparison to seed tubers of potato and also require less input of labor in the field. Further the sugar beet is also more frost tolerant than potato and could result in a longer campaign period for the production of extracted compounds. This last part is very important in a field where infrastructure for production is utilized during a limited time i.e. autumn for potato starch production, and an increased campaign period would increase the utilization and cost efficiency of production facilities.

Thus sugar beet producing starch in the tap-roots instead of sucrose would be a superior alternative to potato for the production of starch in facilities currently used for potato starch production. From a physiological perspective potatoes and sugar beet for starch production could be processed in the same facilities with some modifications.

Starch has many important applications for food as well as for technical purposes. To this end in order to optimize the utility of starch for various applications it is physically or chemically modified. Main use of starch in the food industry is as a thickener and for coating of food products. In technical applications large amounts of starch is used in the paper industry as well as in the textile industry. Other uses are in dispersions, adhesives and drilling applications.

Starch is found as small granules which form and size depend on botanical origin. Starch is a polymer of glucose residues and is a mixture of two distinct components or molecules, amylopectin and amylose. Amylopectin is a very large branched molecule and amylose is considerably smaller and essentially linear. Both contain the same chemical linkages between the glucose residues. Commonly root or tuber starches are composed of 75-80% amylopectin and 20-25% amylose by weight. Starch is a very common storage compound among expanded primary roots and tubers although the absolute amounts out of fresh or dry weight may vary depending on source. Starch can be stained by iodine and this staining is readily visualized by the naked eye or using a microscope. Uncommon among tap-root and tuber crops is sugar beet in that nor can any staining by iodine be seen and neither can any starch structures be visualized under a microscope. Starch is formed in plastids which are subcellular organelles. In photosynthetic cells these are termed choloroplasts while in heterotrophic organs they are termed amyloplasts although starch is formed in both differentiations of plastid organelles. In dicotyledonous plants, glucose-6-phosphate is imported into the amyloplast and subsequently converted to glucose-1-phosphate by plastidic phosphoglucomutase. Glucose-1-phosphate is then converted to ADP-glucose by ADP-glucose pyrophosphorylase using ATP with PPi as a by-product. In plants ADP-glucose pyrophosphorylase is a heterotetramer consisting of two different subunits, one large and one small. Different soluble starch synthases polymerize ADP-glucose into α-1,4 linked glucose residues. The different forms of soluble starch synthase have been shown to be responsible for different chain lengths in the amylopectin. A starch synthase bound to starch is responsible for the synthesis of the long α-1,4 chains of amylose. Starch branching enzymes are responsible for the α-1,6 linkages of especially amylopectin via breaking of a chain at an α-1,4 linkage and attaching it in an α-1,6 position at a different site. Thus no new net production of starch is caused by starch branching enzyme but only a rearrangement. In order for the starch molecules or more specifically the amylopectin to be arranged into the ordered structures of a starch granule, isoamylases have been shown to be of importance for this ordered assembly.

### SUMMARY OF THE INVENTION

The object of the present invention is to produce starch in the tap-root of Beta vulgaris subspecies such as sugar beet, fodder beet and sea beet. Starch have until now not been demonstrated to be produced in the tap-root of Beta vulgaris which normally is used for the production of sugars primarily in the form of sucrose.

The invention relates in one aspect to a genetically modified Beta vulgaris subspecies sugar beet, fodder beet or sea beet plant having starch accumulation in the tap-root. By use of genetic engineering and introducing new genes as well as directing the corresponding polypeptides to the plastids it is for the first time possible to produce starch in the tap root of Beta vulgaris. Thus it is for the first time possible to grow Beta vulgaris in the field for starch production which makes starch extraction more flexible and possible during longer periods of the year in the industry which utilizes potato for starch extraction. The use of Beta vulgaris in this production furthermore has the advantages of reducing inputs into cultivation in the form of labour, water and chemicals.

In a second aspect, the invention relates to a genetically modified Beta vulgaris plant cell comprising at least one heterologous gene selected from the group consisting of SEQ ID NO:1, 3, 5, 7, 9, 12 or 14 or a gene having 70, 75, 80, 85, 90, 95 or 99% identity to SEQ ID NO:1, 3, 5, 7, 9, 12 or 14.

In a third aspect the invention relates to a genetically modified Beta vulgaris plant cells encoding at least one polypeptide selected from the group consisting or SEQ ID NO:2, 4, 6, 8 or 10 or a heterologous polypeptide having 70, 75, 80, 85, 90, 95 or 99% % identify to SEQ ID NO:2, 4, 6, 8 or 10.

In a fourth aspect the invention relates to a method of manufacturing starch from a genetically modified Beta vulgaris according to any of preceding claims having starch accumulation in the tap-root comprising extracting the starch from the tap-root.

In a fifth aspect the invention relates to starch obtained from the genetically modified Beta vulgaris as defined above.

In a final aspect the invention relates to the use of the obtained starch in technical and food applications.

Further advantages and objects with the present invention will be described in more detail, inter alia with reference to the accompanying drawings.

### BRIEF DESCRIPTION OF THE DRAWINGS

FIG. 1 shows a generic plastid with flow of carbon transport and transformation from glucose-6-phosphate to starch including energy import needed for starch biosynthesis. Explanation of abbreviations of functions of the invention; GPT=G-6-P/Pi antiporter, pPGM=plastidic phosphoglucomutase, AGPase=ADP-glucose pyrophosphorylase, PPa6=plastidic inorganic pyrophosphatase and NTT=ATP/ADP translocator.

FIG. 2 shows a vector map containing a Solanum tuberosum PPa6 gene, produced by using recombination which is indicated by present attB sites.

FIG. 3 shows a vector map containing a Solanum tuberosum NTT1 gene, produced by using recombination which is indicated by present attB sites.

FIG. 4 shows a vector map containing Solanum tuberosum PPa6 and NTT 1 genes, produced by double recombination indicated by present attB sites.

FIG. 5 shows a process for the manufacturing of starch.

FIG. 6 shows light microscopy of tissue sections of transgenic sugar beet tap root cells showing production of starch granules. Starch granules are indicated by arrows.

FIG. 7 shows light microscopy of crushed transgenic sugar beet tap root tissue stained with Lugol's solution showing staining of produced starch granules. Starch granules are indicated by arrows.

### DETAILED DESCRIPTION OF THE INVENTION

### Definitions

In the context of the present application and invention, the following definitions apply:

The term “tap-root” is intended to mean an enlarged, somewhat straight to tapering plant root that grows downward. It forms a center from which other roots sprout laterally.

The term “genetic modification” means the introduction of homologous and/or heterologous foreign nucleic acid molecules into the genome of a plant cell or into the genome of a plant, wherein said introduction of these molecules leads to an accumulation of starch in the tap-root of a developed plant.

The term “heterologous” as used herein describes a relationship between two or more elements which indicates that the elements are not normally found in proximity to one another in nature. Thus, for example, a polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). An example of a heterologous polypeptide is a polypeptide expressed from a recombinant polynucleotide in a transgenic organism. Heterologous polynucleotides and polypeptides are forms of recombinant molecules.

### Beta Vulgaris

Beta vulgaris, for example sugar beet does, as other plant species, produce starch in green tissue when photosynthesis is more active and more sucrose is produced in source tissues than can be utilized in sink tissues. This starch is stored as granules in the same way as more long term storage starch but is degraded during every dark period as part of the diurnal cycle. In view of the lack of starch in sugar beet tap-root it could be assumed that some central activity of starch synthesis or assembly is lacking in sugar beet tap-root. Parsnip is a root crop which largely stores starch but also to some extent sugars in the primary enlarged root and was chosen as a relevant comparator with regards to what starch biosynthetic activities could be detected in sugar beet and to what ratio they were manifested.

Our microscopic analysis of tap-root under development revealed a common first state of development which also can be seen in other underground storage tissues. That is cells which are filled by an expanded vacuole containing sugars. With development, parsnip and other starch storing underground tissues will initially form small starch granules which are displaced to the fringes of the cell by the vacuole. Later in development the starch granules continue to grow on the expense of the room available for the vacuole. As a contrast sugar beet tap-root cells display an essentially unchanged cell structure all through development with a large vacuole. Thus sugar beet storage cells seemingly remain in a juvenile state all through development. Since starch is produced to some extent in known underground storage organs it could be speculated that there is some deficiency of a core enzymatic activity in sugar beet tap-root which results in a complete lack of starch production.

Investigation of core biosynthetic activities as ADP-glucose pyrophosphorylase, soluble starch synthase and branching enzyme activities were assayed and compared. Surprisingly enzyme activities essential for the production of starch could be found to be manifested in sugar beet tap-root and furthermore at a level in the same range as in a comparable organ of a starch storing crop. Thus there is starch biosynthetic machinery available in the sugar beet tap-root which for other reasons than core biosynthetic activities is not channeling exported carbon from source tissues into starch in the sink tissue but only sucrose.

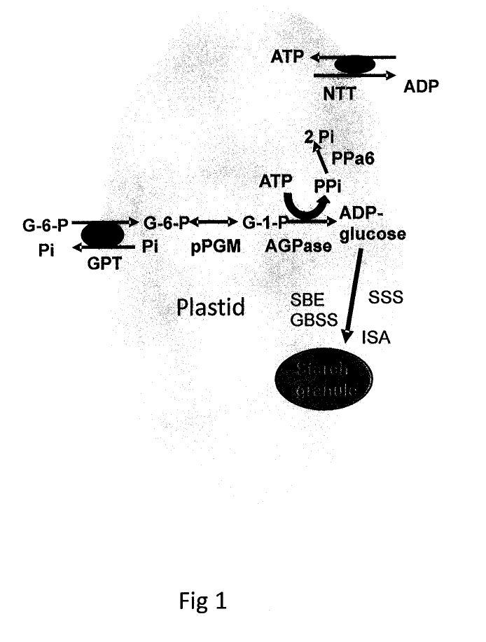

Transcriptome analysis of sugar beet tap-root under development was performed. This was set up with the parsnip tap-root transcriptome being used as a comparator since parsnip deposits starch as well as sugars in the rap root during development. What could be noted from this analysis was that most starch biosynthetic genes were expressed at a lower level but this could not explain the complete absence of starch. This is also supported by the different enzyme assays which we found to be in the same range for both species. Most other genes that code for enzymes involved in metabolic processing of sugars were found to be expressed in both species. A number of genes which products exert or could be envisioned to exert control points with regards to the accumulation of starch were identified and which expression to various extents promote starch accumulation. Out of these genes, five could clearly be identified as having a vastly lower transcript level in a developmental stage of sugar beet tap-root as compared to that of parsnip tap-root, these five functions are highlighted in FIG. 1.

### Genes and Enzymes for the Production of Starch

Starch is produced in special organelles called plastids. Generally genes and encoded enzymes contain a signal sequence of importance for targeting an enzyme to the plastid. For a person skilled in the art it is clear that this signal sequence could be exchanged for other signal sequences targeting the protein providing a specific function to the plastid. The examination of databases, such as are made available, for example, by the EMBL website (see Toolbox at the EBI) or the NCBI (National Center for Biotechnology Information) website, can also be used for identifying homologous sequences to the genes mentioned below, which code for the different polypeptides mentioned below. In this case, one or more sequences are specified as a so-called query. This query sequence is then compared by means of statistical computer programs with sequences, which are contained in the selected databases. Such database queries (e.g. blast or fasta searches) are known to the person skilled in the art and can be carried out by various providers.

If such a database query is carried out, e.g. at the NCBI (National Center for Biotechnology Information) website, then the standard settings, which are specified for the particular comparison inquiry, should be used. For protein sequence comparisons (blastp), these are the following settings: Limit entrez=not activated; Filter=low complexity activated; Expect value=10; word size=3; Matrix=BLOSUM62; Gap costs: Existence=11, Extension=1.

For nucleic acid sequence comparisons (blastn), the following parameters must be set: Limit entrez=not activated; Filter=low complexity activated; Expect value=10; word size=11.

With such a database search, the sequences described in the present invention can be used as a query sequence in order to identify further nucleic acid molecules and/or proteins, providing functions which could be used to accumulate starch in the tap-root of Beta vulgaris.

With the help of the described methods, it is also possible to identify and/or isolate nucleic acid molecules according to the invention, which hybridise with the sequence specified under SEQ ID NO 1, 3, 5, 7, 9, 12 and 14, which encodes different polypetides which are mentioned below.

Within the framework of the present invention, the term “hybridising” means hybridisation under conventional hybridisation conditions, preferably under stringent conditions such as, for example, are described in Sambrock et al., Molecular Cloning, A Laboratory Manual, 3rd edition (2001) Cold Spring Harbor Laboratory Press, Cold Spring Harbor, N.Y. ISBN: 0879695773, Ausubel et al., Short Protocols in Molecular Biology, John Wiley & Sons; 5th edition (2002), ISBN: 0471250929). Particularly preferably, “hybridising” means hybridisation under the following conditions:

### Hybridisation Buffer:

2.times.SSC; 10.times.Denhardt solution (Ficoll 400+PEG+BSA; Ratio 1:1:1); 0.1% SDS; 5 mM EDTA; 50 mM Na2HPO4; 250 μg/ml herring sperm DNA; 50 μg/ml tRNA; or 25 M sodium phosphate buffer pH 7.2; 1 mM EDTA; 7% SDS Hybridisation Temperature: T=65 to 68.degree. C. Wash buffer: 0.1.times.SSC; 0.1% SDS Wash temperature: T=65 to 68.degree. C.

In principle, nucleic acid molecules, which hybridise with the nucleic acid molecules according to the invention, can originate from any plant species, which codes a protein providing an appropriate function, preferably they originate from starch-storing plants and are expressed in underground storage organs although if the same function is provided its origin is not of importance. Nucleic acid molecules, which hybridise with the molecules according to the invention, can, for example, be isolated from genomic or from cDNA libraries. The identification and isolation of nucleic acid molecules of this type can be carried out using the nucleic acid molecules according to the invention or parts of these molecules or the reverse complements of these molecules, e.g. by means of hybridisation according to standard methods (see, for example, Sambrook et al., Molecular Cloning, A Laboratory Manual, 3rd edition (2001) Cold Spring Harbor Laboratory Press, Cold Spring Harbor, N.Y. ISBN: 0879695773, Ausubel et al., Short Protocols in Molecular Biology, John Wiley & Sons; 5th edition (2002), ISBN: 0471250929) or by amplification using PCR.

Nucleic acid molecules, which exactly or essentially have the nucleotide sequence specified under SEQ ID NO 1, 3, 5, 7, 9, 12 and 14 or parts of these sequences, can be used as hybridisation samples. The fragments used as hybridisation samples can also be synthetic fragments or oligonucleotides, which have been manufactured using established synthesising techniques and the sequence of which corresponds essentially with that of a nucleic acid molecule according to the invention.

In conjunction with the present invention, the term “identity” means a sequence identity over the whole length of the coding region less any sequence coding for targeting signals of at least 70%, such as 85%, 90%, 95%, 96%, 97%, 98% or 99%. In conjunction with the present invention, the term “identity” is to be understood to mean the number of amino acids/nucleotides (identity) corresponding with other proteins/nucleic acids, expressed as a percentage. Identity is preferably determined by comparing SEQ ID NO 2, 4, 6, 8, 10, 11, 13 or 15 for amino acids or SEQ. 10 NO 1, 3, 5, 7, 9, 12 or 14 for nucleic acids with other proteins/nucleic acids with the help of computer programs. If sequences that are compared with one another have different lengths, the identity is to be determined in such a way that the number of amino acids, which have the shorter sequence in common with the longer sequence, determines the percentage quotient of the identity. Preferably, identity is determined by means of the computer program ClustalW, which is well known and available to the public (Thompson et al., Nucleic Acids Research 22 (1994), 4673-4680). ClustalW is made publicly available by Julie Thompson (Thompson@EMBL-Heidelberg.DE) and Toby Gibson (Gibson@EMBL-Heidelberg.DE), European Molecular Biology Laboratory, Meyerhofstrasse 1, D 69117 Heidelberg, Germany. ClustalW can also be downloaded from different Internet sites, including the IGBMC (Institut de Genetique et de Biologie Moleculaire et Cellulaire, B.P.163, 67404 Illkirch Cedex, France; ftp://ftp-igbmc.u-strasbg.fr/pub/) and the EBI

(ftp://ftp.ebi.ac.uk/pub/software/) as well as from all mirrored Internet sites of the EBI (European Bioinformatics Institute, Wellcome Trust Genome Campus, Hinxton, Cambridge CB10 1SD, UK).
I. Glucose-6-phosphate/phosphate translocator (SEQ ID NO:1 and 2)

In one aspect the invention relates to a genetically modified Beta vulgaris, such as sugar beet, fodder beet or sea beet which have one of more genes that have been introduced into the plant and plant cells, wherein the introduced gene is involved in the production of starch. One of the genes may be a gene encoding a Glucose-6-phosphate/phosphate translocator shown in SEQ ID NO:1 and 2 or a heterologous gene or peptide having 70, 75, 80, 85, 90, 95, 96, 97, 98 or 99% identity to SEQ ID NO:1 or 2. The Glucose-6-phosphate/phosphate translocator is active at the plastid organelle membrane in an antiporter activity importing hexose phosphate into the plastid in exchange for phosphate. This translocator is of importance for the import of glucose-6-phosphate into the plastid where glucose-6-phosphate is an essential precursor for starch biosynthesis in heterotrophic organs. Two forms are expressed in parsnip while importantly only one is expressed in sugar beet tap-root.

### II. Plastidic Phosphoglucomutase (SEQ ID NO:3, 4 and 11)

In another aspect the invention relates to a genetically modified Beta vulgaris, such as sugar beet, fodder beet or sea beet which have one of more genes that have been introduced into the plant and plant cells, wherein the introduced gene is involved in the production of starch. One of the genes may be a gene encoding a Plastidic phosphoglucomutase shown in SEQ ID NO:3, 4 and 11 or a heterologous gene or peptide having 70, 75, 80, 85, 90, 95, 96, 97, 98 or 99% identity to SEQ ID NO: 2,3 or 11. Plastidic phoshoglucomutase catalyzes the inter conversion of glucose-6-phosphate and glucose-1-phosphate via phosphotransferase activity. The plastidic phosphoglucomutase can thus transform glucose-6-phosphate imported into the plastid into glucose-1-phosphate which is a precursor downstream of glucose-6-phosphate in starch biosynthesis. The ratio between plastidic phosphoglucomutase expression in parsnip compared to sugar beet was found to be high.

### III. Large Subunit of ADP-Glucose Pyrophosphorylase (SEQ ID NO:5 and 6)

In another aspect the invention relates to a genetically modified Beta vulgaris, such as sugar beet, fodder beet or sea beet which have one of more genes that have been introduced into the plant and plant cells, wherein the introduced gene is involved in the production of starch. One of the genes may be a gene encoding a large subunit of ADP-glucose pyrophosphorylase shown in SEQ ID NO:5 and 6 or a heterologous gene or peptide having 70, 75, 80, 85, 90, 95, 96, 97, 98 or 99% identity to SEQ ID NO: 5 and 6. ADP-glucose pyrophosphorylase catalyzes the production of ADP-glucose using glucose-1-phosphate and ATP as substrates. This enzymatic step provides the immediate activated sugar substrate for starch biosynthesis and is seen as the first committed step of starch biosynthesis. In plants ADP-glucose pyrophosphorylase is a hetero tetramer of 2 large subunits and 2 small subunits although the genes coding for both types of subunits contain extensive homology. Two different forms of large subunits were found to be expressed in tap-roots of both species. Both the large subunit forms in sugar beet were found to be expressed at a rather low level as was one form in parsnip while one form in parsnip was found to be very highly expressed and at a much higher level than the most highly expressed large subunit form in sugar beet.

### IV. ATP/ADP Translocator (SEQ ID NO:7, 8, 12 and 13)

In another aspect the invention relates to a genetically modified Beta vulgaris, such as sugar beet, fodder beet or sea beet which have one of more genes that have been introduced into the plant and plant cells, wherein the introduced gene is involved in the production of starch. One of the genes may be a gene encoding a ATP/ADP translocator shown in SEQ ID NO:7, 8, 12 and 13 or a heterologous gene or peptide having 70, 75, 80, 85, 90, 95, 96, 97, 98 or 99% identity to SEQ ID NO: 7, 8, 12 and 13. The ATP/ADP translocator provides energy in the form of ATP to the plastid in a counter exchange of ADP at the plastid membrane. ATP is needed by ADP-glucose pyrophosphorylase in the production of the activated sugar ADP-glucose which is an immediate substrate for starch biosynthesis via starch synthases. Two different but very closely related forms were found to be expressed in parsnip tap-root with one form at a very low level. One form of the ATP/ADP translocator was found to be expressed in sugar beet. The ratio of expression of ATP/ADP translocator between the two species was found to be very high with the higher expression in parsnip.

### V. Plastidic Inorganic Pyrophosphatase (SEQ ID NO:9, 10, 14 and 15)

In another aspect the invention relates to a genetically modified Beta vulgaris, such as sugar beet, fodder beet or sea beet which have one of more genes that have been introduced into the plant and plant cells, wherein the introduced gene is involved in the production of starch. One of the genes may be a gene encoding a plastidic inorganic pyrophosphatase shown in SEQ ID NO:9, 10, 14 and 15 or a heterologous gene or peptide having 70, 75, 80, 85, 90, 95, 96, 97, 98 or 99% identity to SEQ ID NO: 9, 10, 14 and 15. Inorganic pyrophosphatase splits pyrophosphate into two units of inorganic phosphate. Pyrophosphate is a by-product of ADP-glucose production by ADP-glucose pyrophosphorylase. As a by-product phosphate needs to be transported out of the plastid by counter exchange transporters in order to not have an inhibitory effect on starch biosynthesis. One form of the plastidic inorganic pyrophosphatase is expressed in parsnip as well as sugar beet tap-root. In parsnip this gene is expressed at a much higher level as compared to in sugar beet tap-root.

In another aspect the invention relates to a genetically modified Beta vulgaris, such as sugar beet, fodder beet or sea beet which have 1, 2, 3, 4, 5 or more genes introduced into the genome of the plant, wherein said genes are involved in the production of starch. The starch may be visualized by a microscope and/or iodine.

Thus five genes were found of importance for supporting starch biosynthesis and to have a much higher expression in a starch accumulating tap-root under development such as parsnip as compared to the exclusively sucrose accumulating tap-root of sugar beet.

Although the mentioned genes were not found to be completely silent in sugar beet tap-root they were determined to have suboptimal expression and in some examples lack sufficient manifestation to drive starch biosynthesis. In particular expression of the ATP/ADP translocator is needed to supply a heterotrophic organ as sugar beet with sufficient energy for starch biosynthesis and ATP needed for the production of ADP-glucose by ADP-glucose pyrophosphorylase which forms the first committed step in the biosynthesis of starch.

Identified genes code for enzymes and transporters providing functions of importance for the onset of starch accumulation in sugar beet tap-root. Onset of starch synthesis could be accomplished by up regulation in the appropriate tissue of native Beta vulgaris genes providing the identified functions. However genes providing the identified functions can also be isolated from other sources and expressed in the appropriate tissue of sugar beet. One obvious source from our performed studies would be parsnip. Another source of genes providing mentioned functions could be potato. There could be a difference in functional efficiency of said functions depending of gene source. Genes providing enzymes and functions already in operation in underground storage tissues such as potato could be a preferable source although desired effects with regards to onset of starch accumulation in sugar beet tap-root could be provided by genes providing the same functions from other sources. The selection of gene source for these functions are not limited to potato but genes coding for enzymes of a corresponding enzymatic function and localization could be isolated from other organisms. Selection of organisms would thus not be limited to plants.

Said functions will on their own when manifested in sugar beet tap-root enhance starch production. When expressed in combination they provide a further enhanced effect in starch production in sugar beet tap-root. Thus each function can provide a solution to the onset of starch accumulation in sugar beet but they will also in combination provide enhanced effect yielding improved ability to extract starch from sugar beet tap-root tissue.

As mentioned a gene coding for a form of plastid ATP/ADP translocator responsible for supplying the plastid with energy corresponding to Arabidopsis NTT1, displayed a very large difference in expression between sugar beet tap-root and parsnip tap-root. Another gene was coding plastid inorganic pyrophosphate which might be responsible for hydrolyzing PPi which is produced as a residual product of ADP-glucose production. Genes corresponding to a plastid ATP/ADP translocator and plastidic inorganic pyrophosphatase were isolated from a potato cDNA library and named StNTT1 and StPPa6 respectively.

### Expression Vectors, Transformation and Analysis of Material

Furthermore, the invention relates to recombinant nucleic acid molecules containing a nucleic acid molecule according to the invention.

In conjunction with the present invention, the term “recombinant nucleic acid molecule”, such as a binary vector is to be understood to mean a nucleic acid molecule, which contains additional sequences in addition to nucleic acid molecules according to the invention, which do not naturally occur in the combination in which they occur in recombinant nucleic acids according to the invention. Here, the abovementioned additional sequences can be any sequences, preferably they are regulatory sequences (promoters, termination signals, enhancers), particularly preferably they are regulatory sequences that are active in plant tissue, and especially particularly preferably they are regulatory sequences that are active in the tap-root of the plant, in which storage starch is synthesised. Methods for the creation of recombinant nucleic acid molecules according to the invention are known to the person skilled in the art, and include genetic methods such as bonding nucleic acid molecules by way of ligation, genetic recombination, or new synthesis of nucleic acid molecules, for example (see e.g. Sambrok et al., Molecular Cloning, A Laboratory Manual, 3rd edition (2001) Cold Spring Harbour Laboratory Press, Cold Spring Harbour, N.Y. ISBN: 0879695773, Ausubel et al., Short Protocols in Molecular Biology, John Wiley & Sons; 5th edition (2002), ISBN: 0471250929).

For example to express desired functions for the onset of starch biosynthesis in sugar beet tap-root, expression of a promoter with high and specific expression in tap-root may be used, such as genes encoding desired functions were fused to the major latex like gene promoter (M11) of sugar beet. Other promoter sequences can also be used either derived from sugar beet or from other species as long as they result in expression of the fused gene in sugar beet tap-root tissue. A specificity of expression to tap-root tissue is preferable although not needed to practice the invention. Examples of promoters which could be of use to practice the invention in addition to the M11 promoter are the Tlp promoter and the SRD1 promoter (Oltmann et al., 2006 and Noh et al., 2012), well known for a person skilled in the art.

Other examples of promoters are, for example, the promoter of the 35S RNA of the cauliflower mosaic virus and the ubiquitin promoter from maize for constitutive expression, the patatin promoter B33 for tuber-specific expression in potatoes, the USP promoter, the phaseolin promoter, promoters of zein genes from maize, glutelin promoter or shrunken-1 promoter.

Furthermore, a termination sequence (polyadenylation signal) can be present, which is used for adding a poly-A tail to the transcript. A function in the stabilisation of the transcripts is ascribed to the poly-A tail. Elements of this type are described in the literature and can be exchanged at will.

Intron sequences can also be present between the promoter and the coding region. Such intron sequences can lead to stability of expression and to increased expression in plants which is well-known for a person skilled in the art.

In an embodiment, the invention relates to host cells, particularly prokaryotic or eukaryotic cells, which were transformed with a nucleic acid molecule according to the invention or with a vector according to the invention, such as a binary vector, as well as host cells, which originate from these types of host cells, and which contain the described nucleic acid molecules according to the invention or vectors.

The host cells can be bacteria cells, such as E. coli or bacteria of the genus Agrobacterium. For example Agrobacterium tumefaciens or Agrobacterium rhizogenes.

Here, the term “transforms” means that the cells according to the invention are genetically modified with a nucleic acid molecule according to the invention, inasmuch as they contain at least one nucleic acid molecule according to the invention in addition to their natural genome. This can occur in the cell freely, possibly as a self-replicating molecule, or it can be stably integrated into the genome of the host cell.

For example agrobacterium transformation is widely used method for sugar beet transformation and generally a preferred vehicle for the introduction of foreign gene material into chromosomes of sugar beet. Other means of transformation, such as biolistic, injection and infiltration could be used for practicing the invention and long as the desired genetic material is stably maintained in the sugar beet.

Heterologous DNA could be maintained transiently in the cell, autonomously replicated or stably inserted either in chromosomal or plastid DNA.

### Transformation

Recombinant nucleic acid molecules/DNA constructs of the invention can be introduced into the genome of the Beta vulgaris by a variety of conventional techniques. Techniques for transforming a wide variety of higher plant species are well known and described in the technical and scientific literature. See, e.g., Payne, Gamborg, Croy, Jones, etc. all supra, as well as, e.g., Weising et al. (1988) Ann. Rev. Genet. 22:421 and U.S. Pat. Nos. 5,889,191, 5,889,190, 5,866,785, 5,589,367 and 5,316,931. Suitable methods of introducing nucleotide sequences into plant cells and subsequent insertion into the plant genome include microinjection, electroporation, Agrobacterium-mediated transformation, direct gene transfer, and ballistic particle acceleration For example, DNAs can be introduced directly into the genomic DNA of a plant cell using techniques such as electroporation and microinjection of plant cell protoplasts, or the DNA constructs can be introduced directly to plant tissue using ballistic methods, such as DNA particle bombardment. Alternatively, the DNA constructs can be combined with suitable T-DNA flanking regions and introduced into a conventional Agrobacterium tumefaciens host vector. The virulence functions of the Agrobacterium host will direct the insertion of the construct and adjacent marker into the plant cell DNA when the plant cell is infected by the bacteria.

For example, Agrobacterium mediated transformation techniques could be used to transfer the sequences of the invention to transgenic plants. Agrobacterium-mediated transformation is widely used for the transformation of dicots.

### Regeneration of Transgenic Plants

Transformed plant cells which are derived by plant transformation techniques, can be cultured to regenerate a whole plant which possesses the transformed genotype (i.e., the nucleotide sequences mentioned above being involved in the synthesis of starch). Such regeneration techniques rely on manipulation of certain phytohormones in a tissue culture growth medium, typically relying on a biocide and/or herbicide marker which has been introduced together with the desired nucleotide sequences. Methods for transformation and regeneration of sugar beet are known in the art and together with transformation described under Example 5. give guidance to the genetic manipulation of sugar beet (Lindsey and Gallois, 1990; Krens et al., 1996; Joersbo et al., 1998; Hisano et al., 2004; Norouzi et al., 2005), WO01/42480, WO02/14523.

Transformed plant cells, calli or explant can be cultured on regeneration medium in the dark for several weeks, generally about 1 to 3 weeks to allow the somatic embryos to mature. Preferred regeneration media include media containing MS salts. The plant cells, calli or explant are then typically cultured on rooting medium in a light/dark cycle until shoots and roots develop. Methods for plant regeneration are known in the art.

Small plantlets can then be transferred to tubes or other suitable containers containing rooting medium and allowed to grow and develop more roots until visual verification. The plants can then be transplanted to soil mixture in pots in the greenhouse.

The regeneration of plants containing the foreign gene introduced by

Agrobacterium can be achieved as described by Horsch et al., Science, 227:1229-1231 (1985) and Fraley et al., Proc. Natl. Acad. Sci. U.S.A., 80:4803 (1983). This procedure typically produces shoots within two to four weeks and these transformant shoots are then transferred to an appropriate root-inducing medium containing the selective agent and an antibiotic to prevent bacterial growth. Transgenic plants of the present invention may be fertile or sterile.

Regeneration can also be obtained from plant callus, explants, organs, or parts thereof Such regeneration techniques are described generally in Klee et al., Ann. Rev. of Plant Phys. 38:467-486 (1987). The regeneration of plants from either single plant protoplasts or various explants is well known in the art. See, for example, Methods for Plant Molecular Biology, A. Weissbach and H. Weissbach, eds., Academic Press, Inc., San Diego, Calif. (1988).

After transformation with Agrobacterium, the explants typically are transferred to selection medium. One of skill will realize that the selection medium depends on the selectable marker that was co-transfected into the explants. After a suitable length of time, transformants will begin to form shoots. After the shoots are about 1-2 cm in length, the shoots should be transferred to a suitable root and shoot medium. Selection pressure should be maintained in the root and shoot medium.

Typically, the transformants will develop roots in about 1-2 weeks and form plantlets. After the plantlets are about 3-5 cm in height, they are placed in sterile soil in fiber pots. Those of skill in the art will realize that different acclimation procedures are used to obtain transformed plants of different species. For example, after developing a root and shoot, cuttings, as well as somatic embryos of transformed plants, are transferred to medium for establishment of plantlets. For a description of selection and regeneration of transformed plants, see, e.g., Dodds and Roberts (1995) Experiments in Plant Tissue Culture, 3.sup.rd Ed., Cambridge University Press.

The transgenic plants of this invention can be characterized either genotypically or phenotypically to determine the presence of the introduced polynucleotide of the invention. Genotypic analysis can be performed by any of a number of well-known techniques, including PCR amplification of genomic DNA and hybridization of genomic DNA with specific labeled probes. Phenotypic analysis includes, e.g., accumulation of starch in the tap-root.

One of skill will recognize that after the expression cassette containing the heterologous new genes is stably incorporated in transgenic plants and confirmed to be operable, it can be introduced into other plants by sexual crossing. Any of a number of standard breeding techniques can be used, depending upon the species to be crossed.

In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self crossed to produce a homozygous inbred plant. The inbred plant produces seed containing the newly introduced heterologous nucleic acid. These seeds can be grown to produce plants that would produce the selected phenotype.

Transgenic plants expressing a selectable marker can be screened for transmission of the introduced nucleic acid sequences, for example, by standard immunoblot and DNA detection techniques. Transgenic lines are also typically evaluated on levels of expression of the heterologous nucleic acid. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. The RNA-positive plants can then be analyzed for protein expression by Western immunoblot analysis using the specifically reactive antibodies of the present invention. In addition, in situ hybridization and immunocytochemistry according to standard protocols can be done using heterologous nucleic acid specific polynucleotide probes and antibodies, respectively, to localize sites of expression within transgenic tissue. Introduced functions can be analysed by means of enzyme assays. Generally, a number of transgenic lines are usually screened for the incorporated nucleic acid to identify and select plants with the most appropriate expression profiles.

Method of Manufacturing Starch from the Tap-Root of the Plant

Furthermore the present invention relates to a method for the manufacture of starch from Beta vulgaris, such as sugar beet, fodder beet or sea beet including the step of extracting the starch from the tap-root of harvested plants according to the invention.

Methods for extracting starches from plants or from starch-storing parts of plants are known to the person skilled in the art. Furthermore, methods for extracting starch from different starch-storing plants are described, e.g. in Starch: Chemistry and Technology (Publisher: Whistler, BeMiller and Paschall (1994), 2nd Edition, Academic Press Inc. London Ltd; ISBN 0-12-746270-8; see e.g. Chapter XII, Page 412-468: Maize and Sorghum Starches: Manufacture; by Watson; Chapter XIII, Page 469-479: Tapioca, Arrowroot and Sago Starches: Manufacture; by Corbishley and Miller; Chapter XIV, Page 479-490: Potato starch: Manufacture and Uses; by Mitch; Chapter XV, Page 491 to 506: Wheat starch: Manufacture, Modification and Uses; by Knight and Oson; and Chapter XVI, Page 507 to 528: Rice starch: Manufacture and Uses; by Rohmer and Klem; Maize starch: Eckhoff et al., Cereal Chem. 73 (1996), 54-57, the extraction of maize starch on an industrial scale is generally achieved by so-called “wet milling”.). Devices, which are in common use in methods for extracting starch from plant material are separators, decanters, hydrocyclones, centrisiles, vacuum filters, hot air dryers, spray dryers and fluid bed dryers.

The invention also relates to the starch that are obtained from the genetically modified Beta vulgaris defined above as well as the use of the starch in technical and food applications.

Following examples are intended to illustrate, but not to limit, the invention in any manner, shape, or form, either explicitly or implicitly.

### EXAMPLES

### Example 1

### Comparison of Transcriptome Data Sets between Parsnip and Sugar Beet Tap-Root

### Total RNA Extraction

Root and leaf tissue from the plant tissue under development of sugar beet at 54 day after planting (DAP) and parsnip at 61 DAP, were homogenized in liquid nitrogen. Total RNA was extracted with Plant RNA Reagent (Invitrogen, Life technologies Ltd). Concentration was measured on a NanoDrop (NanoDrop™ 1000 Spectrophotometer, Thermo Scientific) and quality was confirmed on a 1.2%, E-gel (Invitrogen, Life Technologies Ltd).

cDNA Library Synthesis

DNA sequencing and data processing was provided by Eurofins as a service. Two normalised random primed cDNA libraries were produced from pooled leaf and tap-root mRNA from sugar beet and parsnip respectively. These were subsequently subjected for sequencing using Roche GS FLX Titanium series chemistry at a scale of ½ segment of a full run for each cDNA library. After quality analysis, passed reads were assembled into contigs and contigs collected in one reference file for each sugar beet and parsnip.

Two 3′-fragment cDNA libraries with bar-coded adaptors were produced from tap-root mRNA from sugar beet and parsnip respectively. These were subsequently subjected to sequencing using Illumina HiSeq 2000 technology utilizing one channel in total for both samples.

After quality analysis, passed reads were mapped to the reference files produced for sugar beet and parsnip. The number of reads mapped to each contig yielded an estimate of gene expression corresponding to the particular contig in comparison to number of reads mapped to other contigs.

Transcriptomes of root tissue in an active storing phase, sugar beet (54 DAP) and parsnip (61 DAP), were compared between sugar beet and parsnip. After quality clipping of the Illumina HiSeq 2000 data, 1.62 fold more clean reads were obtained for P. sativa compared to from B. vulgaris tap-root cDNA. This means that there was 1.62 times the reads available to be mapped to the P. sativa GS FLX reference assembly as compared to the assembly derived from B. vulgaris data.

The quota between P. sativa and B. vulgaris reads actually mapped to the respective reference files was 1.68. This demonstrated a consistency between the different sets of reference data and the quality of mapping to the respective sets of reference data. Thus the FIG. 1.68 was used to adjust the mapping data for B. vulgaris in order to be compared to the data derived from P. sativa. This analysis showed that all major genes coding for starch biosynthetic enzymes or genes coding for hexose-phosphate conversion are expressed in sugar beet tap-root even though there is no starch produced.

### Mapped Illumina HiSeq 2000 Reads

| Genes/functions | B. vulgaris | P. sativa |
|---|---|---|
| ATP/ADP translocator form 1 | 150 | 8213 |
| ATP/ADP translocator form 2 | 0 | 1164 |
| plastidic pyrophosphatase | 166 | 2051 |
| GPT form 1 | 3679 | 4234 |
| GPT form 2 | 0 | 10974 |
| plastidic PGM | 92 | 2125 |
| cytosolic PGM | 805 | 1617 |
| AGPase large | 554 | 15366 |
| AGPase small | 1384 | 6592 |
| Soluble starch synthase | 1218 | 1199 |
| Granule bound starch synthase | 860 | 2945 |
| Starch branching enzyme | 1374 | 4867 |

Number of mapped tap-root Illumina HiSeq 2000 reads on assemblies of B. vulgaris and P. sativa corresponding to different cDNAs of genes of potential importance for starch biosynthesis. The result shown for B. vulgaris was multiplied by 1.68 in order to compensate for differences in total mapped reads between B. vulgaris and P. sativa.

### Example 2

### Starch Biosynthetic Enzymes are Active in Sugar Beet Tap-Root

### Phosphoglucomutase

PGM activity was determined in a spectrophotometric coupled assay. Conversion of glucose-1-phosphate (G1P) is catalyzed by PGM and the resulting glucose-6 phosphate (G6P) is subsequently catalyzed by glucose-6-phosphate dehydrogenase to 6-phosphogluconate. In parallel with the second reaction, NADP is reduced to NADPH and the reaction is measured at 340 nm (Daugherty et al., 1975). Extract corresponding to 20 μg crud protein was added to a substrate solution and the change in absorbance at 340 nm was measured after 2, 5, 10, 15 and 25 minutes. A standard curve was made by assaying various concentrations of phosphoglucomutase (Phosphoglucomutase from rabbit muscle, P3397, SIGMA Aldrich) under the same conditions as the samples. The specificity of the assay was tested by excluding G1P from the substrate. Enzyme activity was calculated as G1P converted to G6P (mop by soluble crude protein (ng) per minute.

### ADP-Glucose Pyrophosphorylase

AGPase activity was determined (Fusari C, Demonte A M, Figueroa C M, Aleanzi M, Iglesias AA (2006). Analytical Biochemistry 352: 145-147) on 20 μg crude protein. The samples were measured after 0, 30 and 90 minutes.

AGPase catalyzes the reaction conversion of ATP and G1P to ADP-glucose and pyrophosphate (PPi).The assay measures phosphate after splitting produced PPi by inorganic pyrophosphatase. A standard curve for phosphate was made by mixing various concentrations of KH₂PO₄ with Mg—Am stain and following the measuring procedure as in the assay. Phosphate content in crude protein extract was measured by inactivating the crude enzyme extract at 60° C. for 10 min and then measuring the samples as described for the standard curve. The background content of pyrophosphate was measured by incubating the inactivated crude extract with inorganic pyrophosphatase and then assaying phosphate content same procedure as the standard curve. Enzyme activity was calculated as produced ADP-glucose (nmol) per soluble crude protein (μg) per minute.

The specificity of the assay was examined by excluding G1P and ATP from the substrate both separately and in combination to determine and exclude the cytosolic UDP-glucose pyrophosphorylase activity.

### Soluble Starch Synthase

10 μg crude root protein extract was assayed for starch synthase activity. Activity was calculated by measurements after 0, 30, 60, 90 and 120 minutes. The starch synthase assay was performed as previously described but with a small modification, where amylopectin in the substrate solution was exchanged to glycogen (Abel et al., 1996), The reaction was terminated at 95° C. for 2 minutes, and precipitated and washed according to the protocol and dissolved in 1 ml ddH₂O. Five ml scintillation mix (Ultima-Flo, Packard) was added to 0.5 ml of the dissolved starch product and radioactivity was measured in a liquid scintillation counter (Philips PW 4700). The starch synthase activity was calculated as the amount ADP-glucose converted to starch per minute and μg total protein.

### Starch Branching Enzyme

20 μg crude protein was assayed for starch branching enzyme activity (Hawker et al., 1974). Activity was calculated by measurements after 45 and 90 minutes. Precipitation, dissolving and counting of radioactivity was performed as described in the starch synthase assay. The starch branching enzyme activity was calculated as the amount glucose-1-phosphate converted to branched starch per minute and μg total protein.

TABLE 1
Starch biosynthetic enzyme activity in soluble protein extracts from sugar beet and parsnip tap-roots

| | Enzyme activity | | | |
|---|---|---|---|---|
| | PGM | AGPase | SS | SBE |
| | (units converting | (nmol ADP- | (nmol ADP-glucose | (nmol G1P converted |
| Root tissue and | 1 μmole G1P to G6P | glucose | converted to starch* | to branched starch * |
| developmental | * ng soluble | * μg soluble | μg soluble | μg soluble |
| stage | protein⁻¹, min⁻¹) | protein⁻¹, min⁻¹) | protein⁻¹, min⁻¹) | protein⁻¹, min⁻¹) |
| Parsnip 48 DAP (light) | 0.07 ± 0.01 | 0.005 ± 0.001 | 0.24 ± 0.01 | 0.54 ± 0.02 |
| Parsnip 48 DAP (dark) | 0.06 ± 0.005 | 0.004 ± 0.001 | 0.25 ± 0.01 | 0.50 ± 0.03 |
| Parsnip 61 DAP (light) | 0.05 ± 0.004 | 0.005 ± 0.001 | 0.35 ± 0.02 | 0.65 ± 0.03 |
| Parsnip 61 DAP (dark) | 0.06 ± 0.01 | 0.005 ± 0.002 | 0.26 ± 0.01 | 0.59 ± 0.02 |
| Sugar beet 41 DAP (light) | 0.07 ± 0.01 | 0.006 ± 0.002 | 0.16 ± 0.01 | 0.43 ± 0.05 |
| Sugar beet 41 DAP (dark) | 0.07 ± 0.004 | 0.006 ± 0.002 | 0.09 ± 0.01 | 0.49 ± 0.06 |
| Sugar beet 54 DAP (light) | 0.07 ± 0.01 | 0.005 ± 0.002 | 0.18 ± 0.01 | 0.63 ± 0.05 |
| Sugar beet 54 DAP (dark) | 0.07 ± 0.01 | 0.008 ± 0.002 | 0.15 ± 0.02 | 0.47 ± 0.02 |

The values for the enzyme activities were determined by triplicate measurements from a single extract consisting of a homogenate of 3 pooled roots.

### Example 3

### Isolation of Genes from Potato

Genes encoding functions of interest were isolated from a potato tuber cDNA library by PCR amplification. Oligonucleotides for the amplification of the genes were designed with a forward primer overlapping the start codon in the 5′-end and a reverse primer overlapping the stop codon in the 3′-end of corresponding genes given as SEQ ID 1, 3, 5, 7 and 9.

After amplification and cloning in a vector system using CloneJet PCR Cloning Kit (Fermentas) each gene sequence was sequenced as a quality control to avoid any mutations introduced by PCR. For genes where no sequence was available comparisons were made with regards to aminoacid sequences of corresponding genes from other plant species than potato. When necessary sequences were corrected using specific oligonucleotides and fusing corrected fragment together before another round of DNA sequencing to confirm that desired changes had taken place. This operation resulted in sequences SEQ ID 1, 3, 5, 7 and 9 were available for further use in sugar beet transformation.

### Example 4

### Gene Constructs for Expression in Sugar Beet Tap-Root

A Gateway® Technology (Life Technologies) in combination with PCR fusion technology was used to introduce an efficient system of enabling the combination of different genes encoding identified functions.

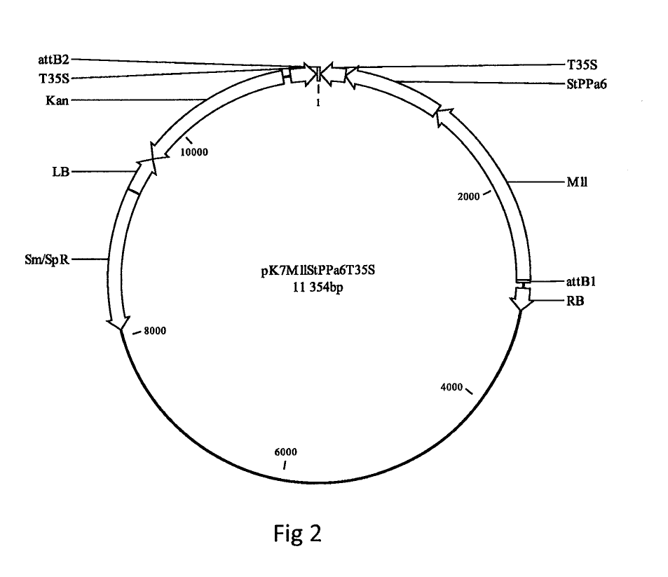

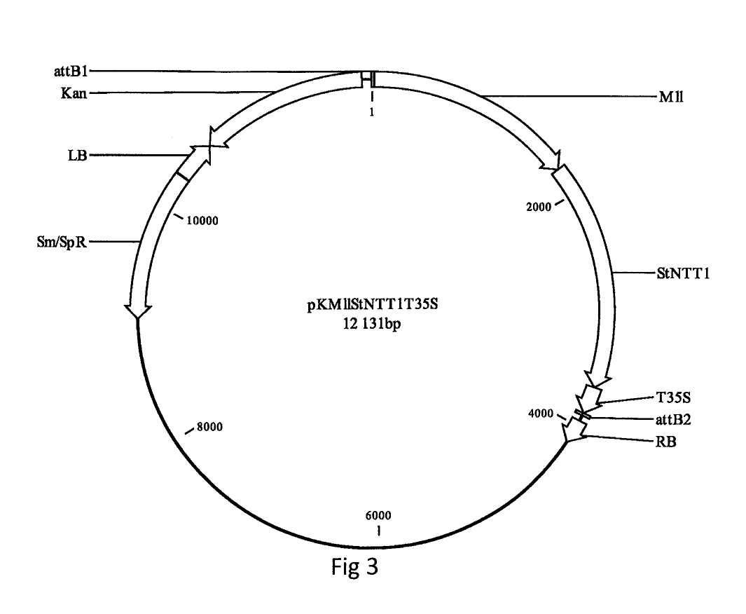

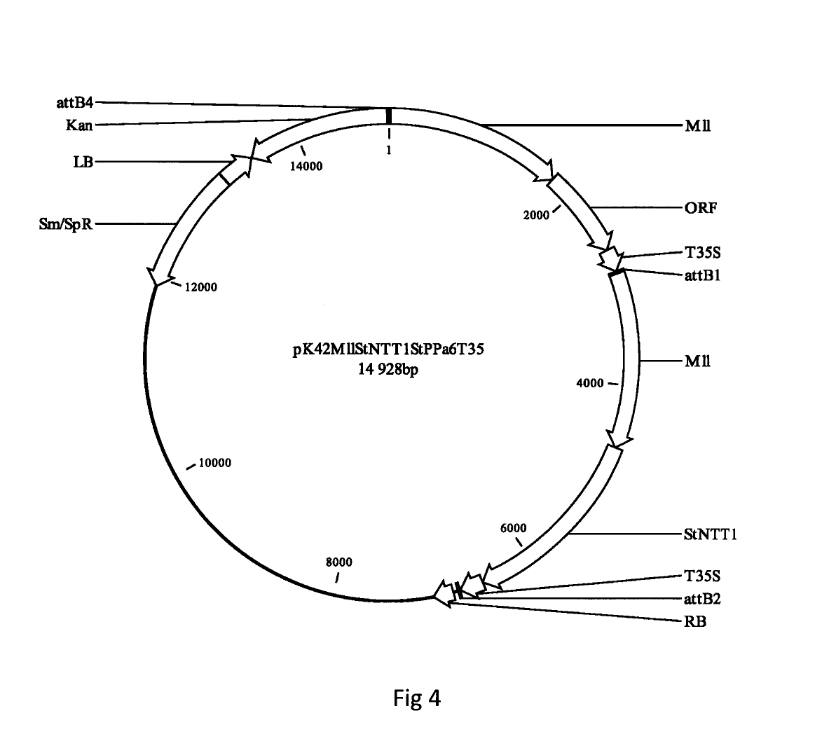

Promoter, gene and terminator combinations were produced by amplification of respective fragments using oligonucleotides with overlapping sequences (20 nucleotide overlap) enabling a subsequent fusion promoter, gene and terminator fragments by annealing via overlapping sequences (40 nucleotides at fusion) and filling in completing a fused gene using a thermostable DNA polymerase, Phusion (Thermo Scientific). In common for all gene constructs created were the M11-promoter and the T35S-terminator. Via the PCR reaction different recombination sites compatible with the MultiSite Gateway® were introduced at the 5′ and the 3′ end of the fused gene constructs. For expression on their own genes were placed in a so called Entry vector and subsequently recombined into a Destination vector via an LR-recombination. Two-gene constructs were made in the same way although upon recombination into the Destination vector one end of both genes need a compatible recombination site while the other end is compatible with the Destination vector in the LR-recombination. Three-gene constructs then were created by one end of gene 1 compatible with one end of gene 2 and the other end of gene 2 compatible with gene 3 and then one end of gene 1 and one end of gene 3 compatible with the Destination vector in an LR-reaction. Larger gene constructs were then iterations of using the same systematic technology. The Destination vector used was in all cases a binary vector suitable for propagation in Agrobacterium tumefaciens and used for transformation of sugar beet. Examples of recombinant nucleic acid molecule made for expressing genes individually, were pK7M11StPPa6T35S (FIG. 2) and pKM11StNTT1T35S (FIG. 3), as well as recombinant nucleic acid molecule for the expression of two genes in sugar beet as pK42M11StNTT1StPPa6T35S (FIG. 4).

### Example 5

### Transformation and Regeneration of Sugar Beet

Agrobacterium tumefaciens harbouring the individual vectors were grown in LB broth supplemented with appropriate antibiotics (50 μg ml⁻¹ rifampicin and 50 μg ml⁻¹ kanamycin or 50 μg ml⁻¹ spectinomycin) at 28° C. over night until an optical density (OD₆₀₀) of 0.6-0.7 is reached. The bacteria was harvested using centrifugation at 4 000×g for 10 min at 4° C. and resuspended in bacterial-induction medium to an OD₆₀₀ of 0.3. The Agrobacterium was grown for additionally 5 h at 28° C. prior inoculation of plant tissues. Leaf explants with exposed shoot base were wounded with a scalpel and immersed in the Agrobacterium suspension for 20 min Excess liquid was drained between two filter papers before the explants were transferred to co-cultivation medium. After 4 days co-cultivation under modest light at 23° C., the explants were rinsed in washing buffer and drained between two filter papers and placed on selection medium with wounded leaf base facing up. Explants were transferred to fresh selection medium every fortnight. Putative transgenic shoots were analysed for the presence of nptII with conventional PCR (S100 termal cycler, Bio-Rad) using REDExtract-N-Amp Plant PCR Kit (Sigma) with primers nptllf 5′-CCTGTCATCTCACCTTGCTC-3′ and nptIIr 5′-AGTCCCGCTCAGAAGAACTC-3′. Transgenic lines were transferred to rooting medium for root formation. As soon as roots were visible the shoots were transferred to MS30 400 claf for continued root development before planted in a phytotron or greenhouse.

### Medium for Sugar Beet Transformation

Bacterial induction medium

| | | |
|---|---|---|
| 0.5* | vitamins | MS salts and B5 |
| 50 | μM | acetosyringone |
| 50 | g l⁻¹ | glucose |
| pH | 5.5 | |

Co-cultivation medium

| | | |
|---|---|---|
| 0.5* | vitamins | MS salts and B5 |
| 50 | μM | acetosyringone |
| 30 | g l⁻¹ | sucrose |
| pH | 5.8 | |
| 8 | g l⁻¹ | phytoagar |

Washing medium

| | | |
|---|---|---|
| 0.5* | vitamins | MS salts and B5 |
| 500 | mg l⁻¹ | claforan |
| pH | 5.8 | |

Selection medium

| | | |
|---|---|---|
| 1* | vitamins | MS salts and B5 |
| 0.25 | mg l⁻¹ | N⁶-benzyladenine |
| 0.10 | mg l⁻¹ | indole-3-butyric acid |
| 400 | mg l⁻¹ | claforan |
| 200 | mg l⁻¹ | kanamycin |
| 30 | g l⁻¹ | sucrose |
| 8 | g l⁻¹ | phytoagar |

Growth medium

| | | |
|---|---|---|
| 1* | vitamins | MS salts and B5 |
| 0.25 | mg l⁻¹ | N⁶-benzyladenine |
| 0.10 | mg l⁻¹ | indole-3-butyric acid |
| 125 | mg l⁻¹ | claforan |
| 30 | g l⁻¹ | sucrose |
| 8 | g l⁻¹ | phytoagar |

### Example 6

### Production of Tap-Root Tissue

After transfer of isolated shoots to rooting medium and the subsequent establishment of roots, shoots were transferred to soil and further propagated in a growth chamber or in the green house. Sugar beet tap-roots were harvested sectioned and flash frozen in liquid nitrogen for subsequent analysis of metabolites and starch.

### Rooting Medium

1*MS salts and B5 vitamins
2.00 mg l^{'1} NAA
1.50 mg l⁻¹ IBA
30 g l⁻¹ sucrose
pH 5.8
6 g l⁻¹ Phytoagar
400 mg l⁻¹ claforan

### Example 7

### Visual Analysis of Starch Deposition

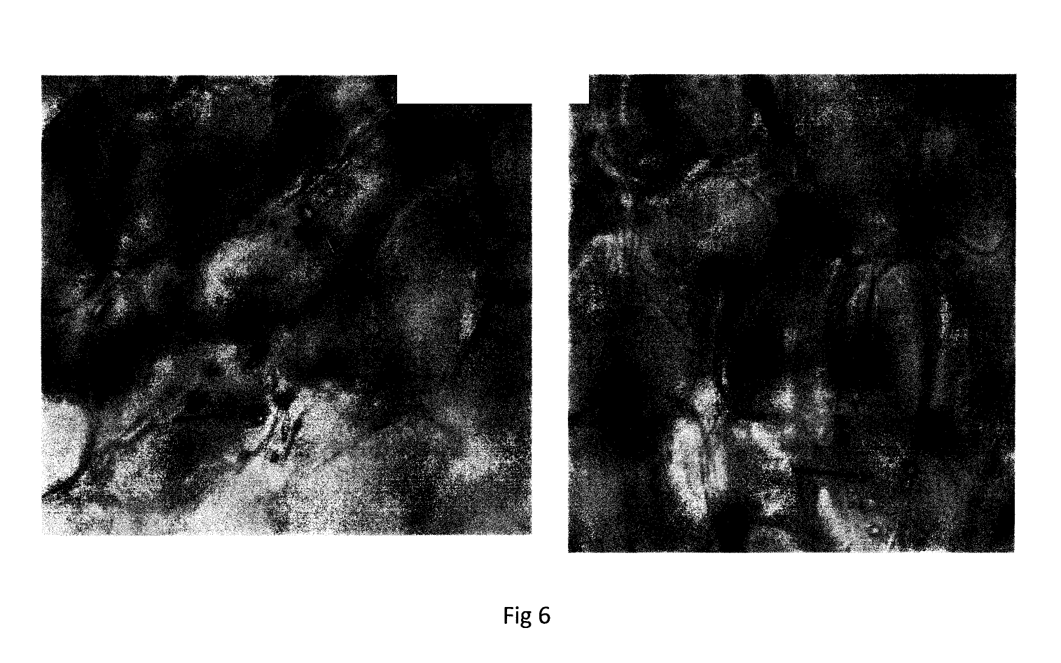

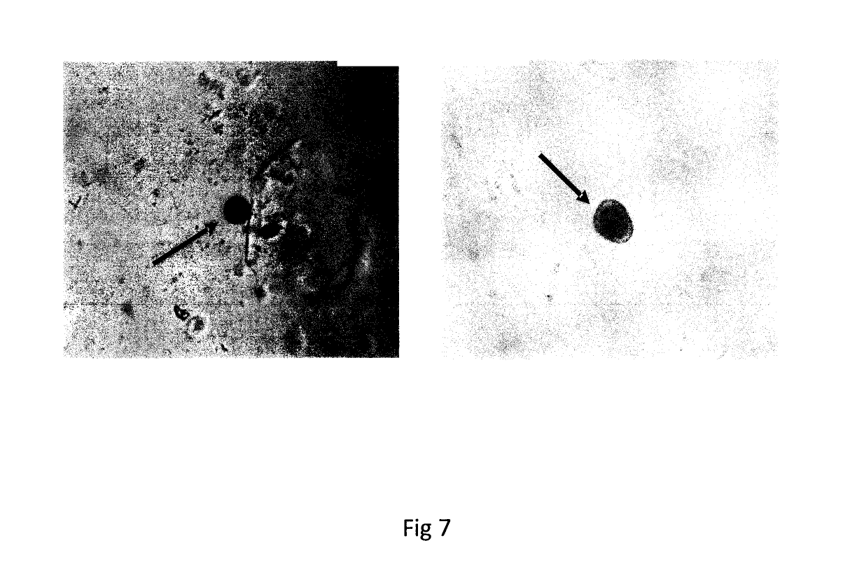

Soil grown sugar beet tap-roots were sectioned and sliced thinly Sections were viewed under a light microscope which revealed starch granules in transgenic sugar beet tap-root sections (FIG. 6). Larger sections of sugar beet tap-root were further homogenized and starch granules stained using Lugol's and visualized under a light microscope (FIG. 7).

### Example 8

### Enzymatic Analysis of Starch

Starch content was analysed using standard method AOAC Method 996.11 and AACC Method 76-13.01, where α-amylase and amyloglucosidase were used for starch digestion following measurement of the released glucose via a glucose oxidase reaction (Total Starch kit, Megazyme). Upon application of the method sugar beet tap-root tissue was found to contain significant amounts of starch in comparison to non-transformed tap-root tissue.

### Example 9

### Purification of Starch

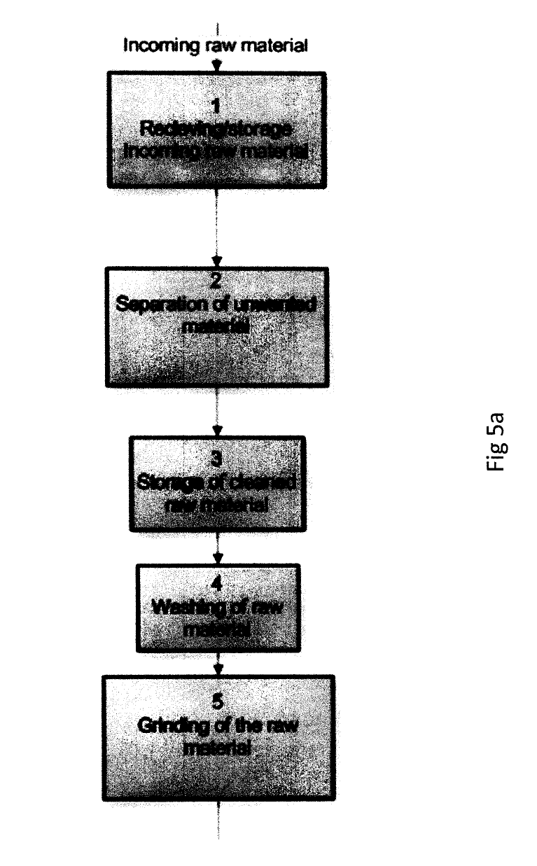

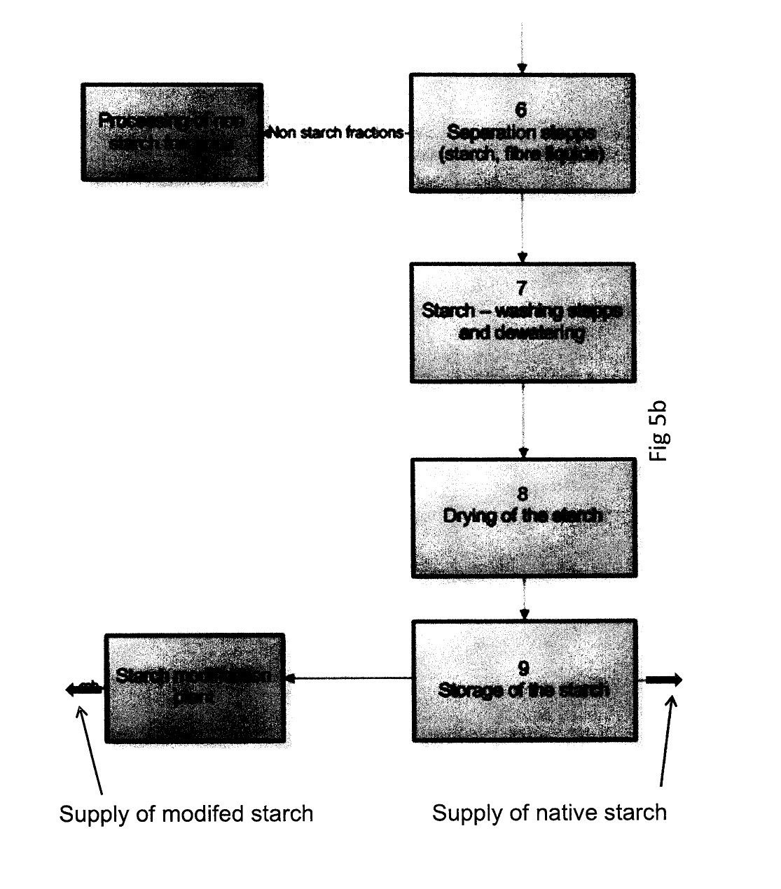

Starch was purified according to FIG. 5.

### REFERENCES

Abel G J W, Springer F, Willmitzer L, Kossmann J (1996) Cloning and functional analysis of a cDNA encoding a novel 139 kDa starch synthase from potato (<i>Solanum tuberosum</i>L.). The Plant Journal 10: 981-991

Daugherty J P, Kraemer W F, Joshi J G (1975) PURIFICATION AND PROPERTIES OF PHOSPHOGLUCOMUTASE FROM FLEISCHMANNS YEAST. European Journal of Biochemistry 57: 115-126

Fusari C, Demonte A M, Figueroa C M, Aleanzi M, Iglesias A A (2006) A colorimetric method for the assay of ADP-glucose pyrophosphorylase. Analytical Biochemistry 352: 145-147

Hawker J S, Ozbun J L, Ozaki H, Greenber.E, Preiss J (1974) INTERACTION OF SPINACH LEAF ADENOSINE-DIPHOSPHATE GLUCOSE ALPHA-1,4-GLUCAN ALPHA-4-GLUCOSYL TRANSFERASE AND ALPHA-1,4-GLUCAN, ALPHA-1,4-GLUCAN-6-GLYCOSYL TRANSFERASE IN SYNTHESIS OF BRANCHED ALPHA-GLUCAN. Archives of Biochemistry and Biophysics 160: 530-551

Hisano H, Kimoto Y, Hayakawa H, Takeichi J, Domae T, Hashimoto R, Abe J, Asano S, Kanazawa A, Shimamoto Y (2004) High frequency Agrobacterium-mediated transformation and plant regeneration via direct shoot formation from leaf explants in Beta vulgaris and Beta maritima. Plant Cell Reports 22: 910-918

Joersbo M, Donaldson I, Kreiberg J, Petersen S G, Brunstedt J, Okkels F T (1998) Analysis of mannose selection used for transformation of sugar beet. Molecular Breeding 4: 111-117

Krens F A, Trifonova A, Keizer L C P, Hall R D (1996) The effect of exogenously-applied phytohormones on gene transfer efficiency in sugarbeet (Beta vulgaris L). Plant Science 116: 97-106

Lindsey K, Gallois P (1990) TRANSFORMATION OF SUGAR-BEET (BETA-VULGARIS) BY AGROBACTERIUM-TUMEFACIENS. Journal of Experimental Botany 41: 529-536

Noh S A, Lee H -S, Huh G H, Oh M -J, Paek K -H, Shin J S, Bae J M (2012) A sweetpotato SRD1 promoter confers strong root-, taproot-, and tuber-specific expression in Arabidopsis, carrot, and potato. Transgenic Research 21: 265-278

Norouzi P, Malboobi M A, Zamani K, Yazdi-Samadi B (2005) Using a competent tissue for efficient transformation of sugarbeet (Beta vulgaris L.). In Vitro Cellular & Developmental Biology-Plant 41: 11-16

Oltmanns H, Kloos D U, Briess W, Pflugmacher M, Stahl D J, Hehl R (2006) Taproot promoters cause tissue specific gene expression within the storage root of sugar beet. Planta 224: 485-495

1. A genetically modified Beta vulgaris subspecies sugar beet, fodder beet or sea beet plant having starch accumulation in the tap-root.

2. The genetically modified Beta vulgaris according to claim 1, wherein the starch being visualised by a microscope and/or iodine.

3. The genetically modified Beta vulgaris according to claim 1, encoding the enzymatic functionality of at least one heterologous polypeptide selected from the group consisting or SEQ ID NO: 2, 4, 6, 8, 10, 11, 13 or 15 or a heterologous polypeptide having 70% identify to SEQ ID NO: 2, 4, 6, 8, 10, 11, 13 or 15 wherein said polynucleotides encodes polypeptides being involved in the synthesis of starch.

4. The genetically modified Beta vulgaris according to claims 3, encoding the heterologous polypeptides SEQ ID NO: 2, 4, 6, 8 or 10 or heterologous polypeptides having 70% identify to SEQ ID NO: 2, 4, 6, 8 or 10.

5. The genetically modified Beta vulgaris according to claim 3, encoding the heterologous polypeptides SEQ ID NO: 2, 6, 11, 13 or 15 or heterologous polypeptides having 70% identify to SEQ ID NO: 2, 6, 11, 13 or 15.

6. The genetically modified Beta vulgaris according to claim 3 encoding one or more polypeptides having 75, 80, 85, 90, 95, 96, 97, 98 or 99% identify to SEQ ID NO: 2, 4, 6, 8, 10, 11, 13 or 15.

7. The genetically modified Beta vulgaris according to claim 1, wherein the plant is sugar beet plant or fodder beet plant.

8. A genetically modified Beta vulgaris subspecies sugar beet, fodder beet or sea beet plant cell, comprising at least one heterologous gene selected from the group consisting of SEQ ID NO: 1, 3, 5, 7, 9, 12 or 14 or a gene having 70, 75, 80, 85, 90, 95 or 99% identity to SEQ ID NO: 1, 3, 5, 7, 9, 12 or 14.

9. The genetically modified Beta vulgaris subspecies sugar beet, fodder beet or sea beet plant cells encoding at least one polypeptide selected from the group consisting or SEQ ID NO: 2, 4, 6, 8 or 10 or a heterologous polypeptide having 70, 75, 80, 85, 90, 95 or 99% identify to SEQ ID NO: 2, 4, 6, 8 or 10.

10. A method of manufacturing starch from a genetically modified Beta vulgaris according to claim 9 having starch accumulation in the tap-root comprising extracting the starch from the tap-root.

11. Starch obtained from the genetically modified Beta vulgaris according to claim 10.

12. Use of the obtained starch according to claim 11 in technical and food applications.

13. A method of manufacturing starch from a genetically modified Beta vulgaris according to claim 1 having starch accumulation in the tap-root comprising extracting the starch from the tap-root.

14. A method of manufacturing starch from a genetically modified Beta vulgaris according to claim 8 having starch accumulation in the tap-root comprising extracting the starch from the tap-root.
